(19) United States
(12) Patent Application Publication
(10) Pub. No.: US 2019/0282806 A1
(43) Pub. Date: Sep. 19, 2019

(54) HIGH-FREQUENCY ELECTRODE FOR MEDICAL DEVICE AND MEDICAL DEVICE

(71) Applicant: OLYMPUS CORPORATION, Tokyo (JP)

(72) Inventors: Kentaro TSUDA, Tokyo (JP); Hiroaki KASAI, Tokyo (JP); Takuya FUJIHARA, Tokyo (JP); Yu MURANO, Tokyo (JP)

(73) Assignee: OLYMPUS CORPORATION, Tokyo (JP)

(21) Appl. No.: 16/432,985

(22) Filed: Jun. 6, 2019

Related U.S. Application Data
(63) Continuation of application No. PCT/JP2018/005976, filed on Feb. 20, 2018.

(30) Foreign Application Priority Data
Apr. 10, 2017 (JP) 2017-077673

(51) Int. Cl. A61N 1/06; A61B 18/14

(52) U.S. Cl. CPC A61N 1/06; H01J 61/0735; A61B 18/14

(57) ABSTRACT

A high-frequency electrode for a medical device includes an electrode base material and an oxide. The electrode base material is made of a metal or an alloy. The oxide is added into the electrode base material. The metal or alloy has a melting point of 2000° C. or higher. The oxide has a particle diameter of 2 μm or more.

The application is a continuation application based on a PCT Patent Application No. PCT/JP2018/005976, filed Feb. 20, 2018, whose priority is claimed on Japanese Patent Application No. 2017-077673 filed Apr. 10, 2017. The content of both the PCT Application and the Japanese Application are incorporated herein by reference.

### FIELD OF THE INVENTION

The present invention relates to a high-frequency electrode for a medical device and a medical device.

### DESCRIPTION OF RELATED ART

Medical devices that release high-frequency power to a biological material are known. These medical devices include a high-frequency electrode for medical devices (hereinafter, may simply be referred to as a “high-frequency electrode”) for the purpose of releasing high-frequency power to a biological tissue. The high-frequency electrode comes into contact with the biological tissue during use. When high-frequency power is released from the high-frequency electrode in contact with the biological tissue to the biological tissue, for example, treatment thereof is possible. Examples of treatment of biological tissue include incision, hemostasis or the like.

When a high-frequency current flows from the high-frequency electrode to the biological tissue, Joule heat is generated. By this phenomenon, the biological tissue is heated. When the biological tissue is exposed to high temperatures, for example, protein components and the like are denatured. As a result, the biological tissue firmly adheres to the high-frequency electrode. For this reason, in a high-frequency electrode for medical devices, there is a strong demand for improvement of the adhesion prevention performance with respect to biological tissue.

For example, a high-frequency treatment tool for an endoscope disclosed in Japanese Patent No. 4296141 has a coating on a protruding portion of a high-frequency, electrode. The coating is made of gold, a platinum group metal or a platinum group alloy. Japanese Patent No. 4296141 discloses that as a result of forming the coating on an electrode surface, oxidation of the electrode surface is prevented. Further, it is disclosed that as a result, adhesion of biological tissue is reduced.

For example, in the high-frequency treatment tool disclosed in Japanese Unexamined Patent Application, First Publication No. 2015-57089, a material having a thermal conductivity of 18 W/m K or more and 30 Wm·K or less at 100° C. is used for an electrode portion in contact with body tissue. The material of the electrode portion is, for example, stainless steel.

### SUMMARY OF THE INVENTION

A high-frequency electrode for a medical device according to the first aspect of the present invention includes an electrode base material made of a metal or an alloy, and an oxide added to the electrode base material, in which the metal or the alloy has a melting point of 2000° C. or higher, and the oxide has a particle diameter of 2 μm or more.

According to a high-frequency electrode for a medical device in the second aspect of the present invention, the particle diameter of the oxide may be equal to or less than 1/100 of a representative length of an electrode shape by an effective electrode region in a narrow direction thereof.

According to a high-frequency electrode for a medical device in the third aspect of the present invention, the electrode base material may contain one or more metallic elements selected from the group consisting of tungsten (W), niobium (Nb), and tantalum (Ta).

According to a high-frequency electrode for a medical device in the fourth aspect of the present invention, a standard energy of formation in the standard state (298.15 K and 105 Pa) of the oxide may be equal to or less than −240 kcal/mol.

A medical device according to the fifth aspect of the present invention includes the aforementioned high-frequency electrode for a medical device.

### BRIEF DESCRIPTION OF THE DRAWINGS

FIG. 1 is a schematic front view showing a schematic configuration of a medical device according to an embodiment of the present invention.

FIG. 2 is a schematic cross-sectional view showing an internal configuration of a high-frequency electrode for a medical device according to an embodiment of the present invention.

FIG. 3 is a schematic perspective view showing a first modified example of the high-frequency electrode for a medical device of the embodiment of the present invention.

FIG. 4 is a schematic perspective view showing a second modified example of the high-frequency electrode for a medical device of the embodiment of the present invention.

FIG. 5 is a schematic perspective view showing a third modified example of the high-frequency electrode for a medical device of the embodiment of the present invention.

### DETAILED DESCRIPTION OF THE INVENTION

Hereinafter, a high-frequency electrode for a medical device and a medical device of an embodiment of the present invention will be described.

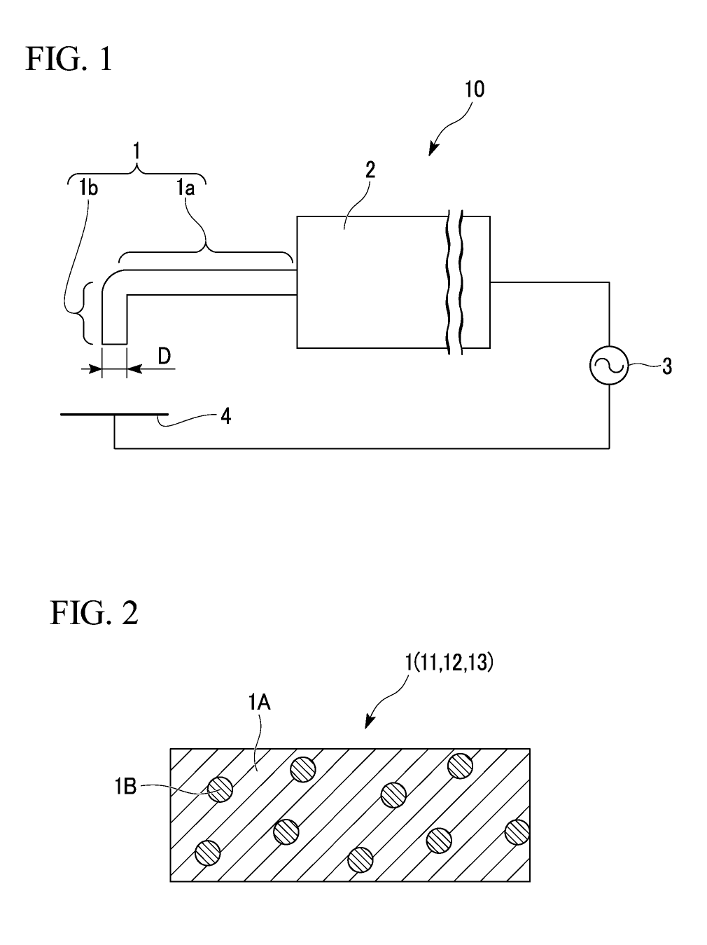

FIG. 1 is a schematic front view showing a schematic configuration of a medical device according to an embodiment of the present invention. FIG. 2 is a schematic cross-sectional view showing an internal configuration of the high-frequency electrode for a medical device of the embodiment of the present invention.

A high-frequency knife 10 of the present embodiment shown in FIG. 1 is an example of the medical device according to the present embodiment.

The high-frequency knife 10 is a medical treatment tool for performing treatment on a biological tissue (a biological material). A high-frequency voltage is applied to the high-frequency knife 10 during use. For example, the high-frequency knife 10 can incise or resect a biological tissue. For example, the high-frequency knife 10 can coagulate (hemostasis) or cauterize a biological tissue.

The high-frequency knife 10 includes a grip portion 2 and a high-frequency, electrode 1 (a high-frequency electrode for a medical device) of the present embodiment. The grip portion 2 has a rod shape that can be held by an operator. The high-frequency electrode 1 protrudes from a tip of the grip portion 2.

The high-frequency electrode 1 comes into contact with biological tissue during use. The biological tissue is a treatment subject. The high-frequency electrode 1 applies a high-frequency voltage to the biological tissue. The high-frequency electrode 1 is electrically connected to a high-frequency power supply 3 via a wiring (not shown). The wiring (not shown) is connected to a proximal end of the high-frequency electrode 1. The high-frequency electrode 1 is supported by the grip portion 2. A counter electrode plate 4 is electrically connected to the high-frequency power supply 3. The counter electrode plate is mounted on the treatment subject.

A shape of the high-frequency electrode 1 is not particularly limited. As the shape of the high-frequency electrode 1, an appropriate shape may be used in accordance with the necessary treatment. In the example shown in FIG. 1, the high-frequency electrode 1 includes, for example, a rod-shaped portion 1a and a hook portion 1b. The rod-shaped portion 1a has a round rod shape. The rod-shaped portion 1a extends in a straight line in a longitudinal direction of the grip portion 2. The hook portion 1b has a round rod shape. The hook portion 1b is a portion bent laterally from a tip of the rod-shaped portion 1a. A bending angle of the hook portion 1b is not particularly limited. In the example shown in FIG. 1, the hook portion 1b is bent in a direction of approximately 90° relative to a longitudinal direction of the rod-shaped portion 1a.

Diameters of the rod-shaped portion 1a and the hook portion 1b in the high-frequency electrode 1 may be the same. The diameters of the rod-shaped portion 1a and the hook portion 1b in the high-frequency electrode 1 can be different from each other. In the following description, as an example, each of the diameters of the rod-shaped portion 1a and the hook portion 1b is represented by D.

FIG. 2 schematically shows a cross-section of the high-frequency electrode 1. As shown in FIG. 2, the high-frequency electrode 1 includes an electrode base material 1A and an oxide 1B. A coat layer (not shown) may be provided on an outer surface of the high-frequency electrode 1. However, at least an effective electrode region on a surface of the high-frequency electrode 1 is not covered by the coat layer. Here, the term “effective electrode region” in the high-frequency electrode 1 means a surface region of the high-frequency electrode 1 where high-frequency power can be released to the biological tissue when the high-frequency electrode 1 is in contact with the biological tissue. As described later, the oxide 1B is not densely aggregated and exposed over a large area on the surface of the high-frequency electrode 1. For this reason, the area where the oxide 1B is exposed on the surface of the high-frequency electrode 1 is also regarded as an effective electrode region.

In the following description, as an example, no coat layer is formed on the surface of the high-frequency electrode 1, and the entire surface of the high-frequency electrode 1 exposed from the grip portion 2 is an effective electrode region.

The electrode base material 1A is made of metal or alloy. The metal or alloy has a melting point of 2000° C. or higher.

Examples of metals having a melting point of 2000° C. or higher include tungsten (W, melting point of 3407° C.), niobium (Nb, melting point of 2467° C.), and tantalum (Ta, melting point of 2996° C.). When the electrode base material 1A is made of an alloy, an appropriate alloy having a melting point of 2,000° C. or higher may be used. For example, an alloy containing one or more metal elements selected from the group consisting of W, Nb, and Ta may be used.

The oxide 1B is added to the electrode base material 1A. The oxide 1B is dispersed in the electrode base material 1A. The oxide 1B has a particle diameter of 2 μm or more. If the particle diameter of the oxide 1B is less than 2 μm, the cooling effect caused by the oxide 1B is reduced.

For the purpose of reducing irregularities in the distribution of the oxide 19 in the electrode base material 1A, the particle diameter of the oxide 1B is preferably equal to or less than 1/100 of a representative length of an electrode shape in the effective electrode region in a narrow direction thereof. The term “narrow direction of the electrode shape in the effective electrode region” and the term “representative length” thereof will be described later.

Oxides having a standard energy of formation equal to or less than −240 kcal/mol in the standard state (298.15 K and 105 Pa) are preferably used for the oxide 1B. Specific oxides having a standard energy of formation equal to or less than −240 kcal/mol include, for example, ThO₂ (thorium dioxide, −279.21 kcal/mol), La₂O₃ (lanthanum oxide, −407.50 kcal/mol), Ce₂O₃ (cerium oxide, −407.09 kcal/mol) and the like.

The oxide 1B may consist of one type of oxide. The oxide 1B may consist of a plurality of oxides.

An addition amount of the oxide 1B in the high-frequency electrode 1 is preferably 1 part by mass or more and 20 parts by mass or less with respect to 100 parts by mass of the electrode base material 1A. The amount of the oxide 1B added to the high-frequency electrode 1 is more preferably 1 part by mass or more and 10 parts by mass or less with respect to 100 parts by mass of the electrode base material 1A.

Here, the term “representative length of the electrode shape in the effective electrode region in the narrow direction” will be described.

The term “electrode shape in the effective electrode region” means that only effective electrode region is considered to define the electrode shape. The term “representative length of the electrode shape” means that, to measure the size of the electrode shape, the electrode shape is represented by a rectangular parallelepiped which accommodates and circumscribes the electrode shape. Among many possible rectangular parallelepipeds which accommodates and circumscribes the electrode shape, one which has the shortest side is considered the one representing the electrode shape, and the length of the shortest side is termed as “representative length of the electrode shape in the narrow direction.”

In order to distribute the oxide 1B uniformly in the effective electrode region, it is important that the particle diameter of the oxide 1B be sufficiently small with respect to a dimension representing narrowness in a solid (three-dimensional) electrode shape in the effective electrode region.

The electrode shape in the effective electrode region of the high-frequency electrode used for a medical device is often formed, for example, into a simple three-dimensional shape such as a rod shape or a plate shape. For example, the high-frequency electrode should have a shape that easily comes into contact with biological tissue. For this reason, substantially deep narrowed portions, recessed portions, and holes are not formed in the effective electrode region.

The narrowness in the electrode shape does not depend on whether the electrode shape is bent or not. For example, in a hook type rod-shaped electrode such as the high-frequency electrode 1 shown in FIG. 1, if the diameters of the rod-shaped portion 1a and the hook portion 1b are uniform, a cross-sectional area orthogonal to a central axis is constant even if it is bent.

When a change of the electrode shape is formed only by bending, the dimension representing the narrowness in the electrode shape can be evaluated for each of simple shapes separated by the bending portion. For example, in the case of the high-frequency electrode 1, the effective electrode region is divided into the rod-shaped portion 1a and the hook portion 1b. The rod-shaped portion 1a and the hook portion 1b are each simple round bars. In this case, the direction in which the rod-shaped portion 1a and the hook portion 1b are narrowest is the radial direction. The representative length in the direction in which they are narrowest is the diameter. The diameters D of the rod-shaped portion 1a and the hook portion 1b are equal to each other. For this reason, the narrowness in each of the rod-shaped portion 1a and the hook portion 1b can be evaluated to be the same.

The electrode shape used for the effective electrode region can be divided into simple shapes even if it is bent as described above. The size of a three-dimensional shape using these simple shapes can be described with a combination of representative lengths L1, L2, and L3 (where L1≥L2≥L3) in three directions orthogonal to each other. The representative lengths L1, L2, and L3 correspond to the lengths of three sides orthogonal to each other in a virtual rectangular parallelepiped (hereinafter, circumscribed rectangular parallelepiped) circumscribing the three-dimensional shape of the effective electrode region. However, each representative length also changes depending on how the circumscribed rectangular parallelepiped is set. For this reason, in the setting of the circumscribed rectangular parallelepiped, the setting in which L3 is the smallest is used.

In the present specification, in the electrode shape in the effective electrode region, a direction in which the representative length L3 is measured is referred to as the “narrow direction.”

In the high-frequency electrode 1, the narrow directions of the rod-shaped portion 1a and the hook portion 1b are their radial directions. The particle diameter of the oxide 1B contained in the high-frequency electrode 1 is preferably equal to or less than D/100.

The high-frequency electrode 1 described above is manufactured, for example, by mixing the powder material for the electrode base material 1A and the oxide 1B, and then using a powder metallurgy method.

Next, an action of the high-frequency knife 10 will be described focusing on an action of the high-frequency electrode 1.

The present inventors have observed the conventional high-frequency electrode to which biological tissue is easily adhered, and found that fine irregularities are formed on the electrode surface. According to the study of the present inventors, for example, when irregularities having a maximum height Ry (JIS B 0601-1994) of 10 μm or more are formed on the electrode surface, the biological tissue adheres easily.

The present inventors assume that such irregularities are formed as a result of sparks melting the metal of the electrode surface. Sparks are generated when high-frequency power is released to the biological tissue.

In the high-frequency electrode 1, since a metal or alloy having a melting point of 2000° C. or higher is used for the electrode base material 1A, it is difficult for the electrode base material 1A itself to melt.

However, when sparks caused by the high-frequency power hit the electrode base material 1A, energy is concentrated on a very small area. For this reason, even with a melting point of 2000° C. or higher, melting in very small areas is not completely eliminated.

The present inventors noted that when oxides are added to a metal, an increase in the temperature of the metal can be suppressed by an endothermic reaction of the oxides. The present inventors have conducted intensive investigations regarding adding the oxide 1B to the electrode base material 1A having a high melting point in order to prolong the life of the high-frequency electrode 1.

As a result, it was found that adding the oxide 1B having a particle diameter of 2 μm or more to the electrode base material 1A made of a metal or an alloy having a melting point of 2000° C. or higher leads to good results. The electrode base material 1A to which the oxide 1B having a particle diameter of 2 μm or more is added can significantly suppress deterioration of the electrode surface as compared with a high-frequency electrode made of a metal or an alloy having a melting point of less than 2000° C.

If the particle diameter of the oxide 1B is less than 2 μm, the endothermic effect of any oxide 19 is too small, so that the effect of preventing the melting of the electrode base material 1A becomes insufficient.

The oxide 1B is a nonconductor. For this reason, if the oxide 1B is added in excess, the electrical resistance of the high-frequency electrode 1 increases. If there is too much of the oxide 19 the performance of the electrode may be reduced and Joule heat may be increased. When the added amount of the oxide 1B was set to the more preferable range mentioned above, such performance deterioration was reliably prevented.

If the particle diameter of the oxide 1B is excessively large, intervals between the particles of the oxide 1B in the electrode base material 1A become too wide when a preferable addition amount is used. In this case, unevenness in the distribution of the oxide 1B in the electrode base material 1A is likely to occur. For this reason, locations at which the distribution of the oxide 19 is not dense are difficult to cool. As a result, irregularities may be easily formed on the surface of the high-frequency electrode 1. When the maximum particle diameter of the oxide 1B falls within the more preferable range mentioned above, such deterioration with time is reliably prevented.

The effect of the endothermic reaction of the oxide 1B is also related to a magnitude of the standard free energy of formation. According to the study results of the present inventors, when materials having a standard free energy of formation in the more preferable range mentioned above are selected as the material of the oxide 1B, a better cooling effect can be obtained. As a result, the formation of fine irregularities on the electrode surface is more reliably suppressed. Although fine irregularities are thought to become more numerous over time due to sparks, such a cooling effect also suppresses an increase in fine irregularities over time.

As described above, according to the high-frequency electrode 1, for example, the generation of irregularities in the electrode surface which is thought to be caused by sparks is suppressed, and as a result, the smoothness of the surface of the high-frequency electrode 1 is easily maintained over time. For this reason, deterioration of the adhesion prevention performance with respect to biological tissue in the high-frequency electrode 1 over time is suppressed. As a result, the treatment performance of the high-frequency electrode 1 is maintained for a long time.

### MODIFIED EXAMPLES

Next, modified examples of the electrode shape of the high-frequency electrode will be described. These modified examples can be used in place of a part or all of the high-frequency electrode 1 in the high-frequency knife 10.

The electrode shape of the high-frequency electrode in the high-frequency knife 10 can be appropriately selected in accordance with the necessity of treatments using the high-frequency knife 10.

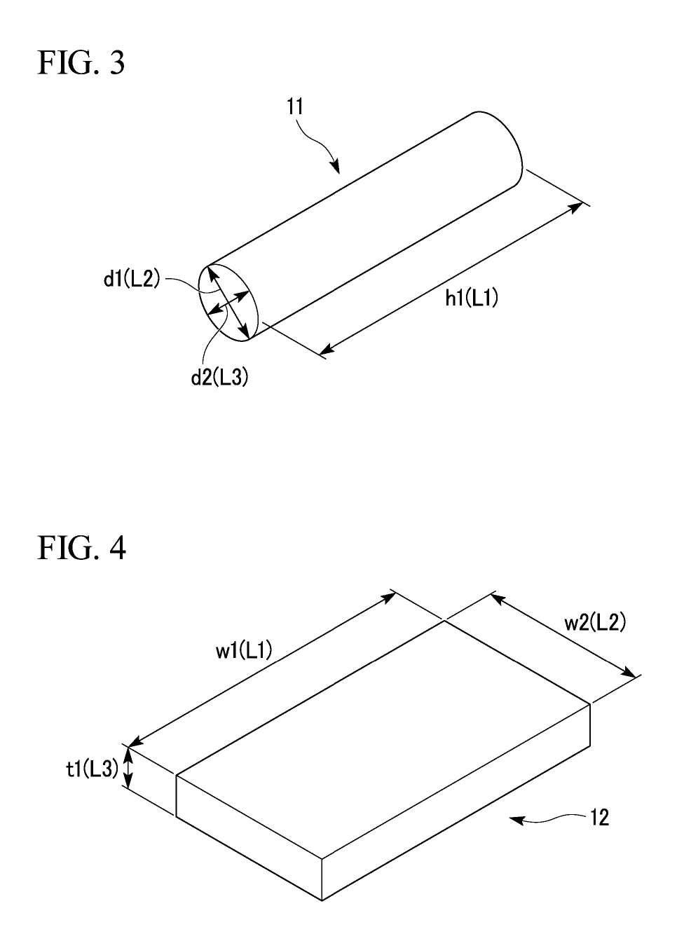

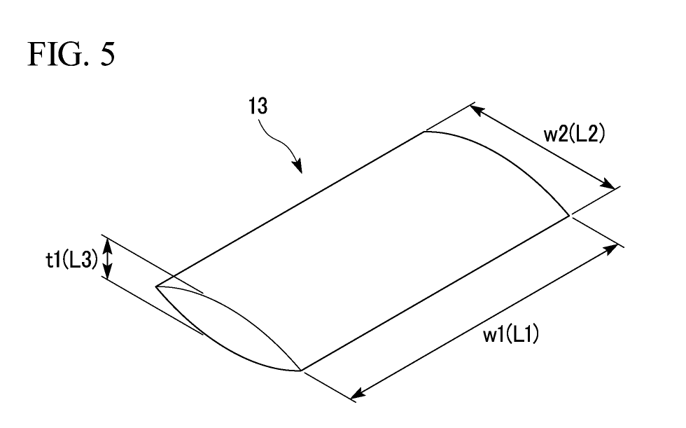

FIG. 3 is a schematic perspective view showing a first modified example of the high-frequency electrode for a medical device of the embodiment of the present invention. FIG. 4 is a schematic perspective view showing a second modified example of the high-frequency electrode for a medical device of the embodiment of the present invention. FIG. 5 is a schematic perspective view showing a third modified example of the high-frequency electrode for a medical device of the embodiment of the present invention.

Each of high-frequency electrodes in the respective modified examples described below includes an electrode base material 1A and an oxide 1B as in the high-frequency electrode 1 of the above embodiment (see FIG. 2). However, the more preferable maximum diameter of the oxide 1B varies in accordance with each electrode shape.

A high-frequency electrode 11 of the first modified example shown in FIG. 3 is formed of a rod-shaped body. The rod-shaped body has an elliptical cross-section with a long diameter d1×a short diameter d2×a length h1 (where h1>d1>d2). The entire surface of the high-frequency electrode 11 is the effective electrode region.

The narrow direction in the electrode shape of the high-frequency electrode 11 is the short diameter direction. The representative lengths L1, L2 and L3 are equal to h1, d1 and d2, respectively.

The particle diameter of the oxide 1B contained in the high-frequency electrode 11 is more preferably equal to or less than d2/100.

A high-frequency electrode 12 of the second modified example shown in FIG. 4 is formed of a flat plate with a longitudinal width w1×a lateral width w2×a thickness t1 (where w1>w2>t1). The entire surface of the high-frequency electrode 12 is the effective electrode region.

The narrow direction in the electrode shape of the high-frequency electrode 12 is the thickness direction. The representative lengths L1, L2 and L3 are equal to w1, w2 and t1, respectively.

The particle diameter of the oxide 1B contained in the high-frequency electrode 12 is more preferably equal to or less than t1/100.

The high-frequency electrode may be a plate-shaped body of which thickness decreases toward an outer edge thereof.

For example, the high-frequency electrode 13 of the third modified example shown in FIG. 5 is formed of a spatula type plate-shaped body. The high-frequency electrode 13 has a thinner thickness at both ends in the lateral width direction than a thickness at a central portion in the lateral width direction of the high-frequency electrode 12.

The outer edge in the lateral width direction of the high-frequency electrode 13 may be sharp in a V-shape. The outer edge in the lateral width direction of the high-frequency electrode 13 may be rounded. For example, in the high-frequency electrode 11 of the elliptic rod shape shown in FIG. 3, the high-frequency electrode 13 may be a flat elliptical rod. In the flat elliptic rod, an aspect ratio of the short diameter d2 to the long diameter d1 is set to be large.

The electrode shape of the high-frequency electrode 13 has a shape with a longitudinal width w1×a lateral width w2× the maximum thickness t1 (where w1>w2>t1). The entire surface of the high-frequency electrode 13 is the effective electrode region.

The narrow direction in the electrode shape of the high-frequency electrode 13 is the thickness direction, similarly to the high-frequency electrode 12 of the second modified example. The representative lengths L1, L2 and L3 are equal to w1, w2 and t1, respectively.

The particle diameter of the oxide 1B contained in the high-frequency electrode 13 is more preferably equal to or less than t1/100.

### Fourth to Sixth Modified Examples

Although not particularly illustrated, the electrode shape of the high-frequency electrode 11 of the first modified example may be deformed into a cylinder of d2=d1 (the fourth modified example). In the high-frequency electrode of the fourth modified example, the hook portion 1b is removed from the high-frequency electrode 1 of the above embodiment.

Although not particularly illustrated, the electrode shape of the high-frequency electrode 11 of the first modified example may be deformed into an elliptical plate or a circular plate such that the length h1 satisfies the conditions h1<d1, h1<d2, and d1≥d2 (the fifth modified example). In the high-frequency electrode formed of such an elliptical plate or a circular plate, the narrow direction is the length direction. In this case, the representative lengths L1, L2 and L3 are equal to d1, d2 and h1, respectively.

In the high-frequency electrode of the fifth modified example, the particle diameter of the oxide 1B is more preferably h1/100 or less.

Although not particularly illustrated, the high-frequency electrode of the fifth modified example may be further deformed into a plate-shaped body of which thickness gradually decreases from the center to the outer edge (the sixth modified example).

Each of the high-frequency electrodes of the modified examples described above includes the electrode base material 1A and the oxide 1B, so that the adhesion prevention performance of the biological tissue is stabilized as in the high-frequency electrode 1 of the above embodiment.

In the description of the above embodiment and each modified example, the example in which the high-frequency electrode for a medical device is used for the high-frequency knife 10 has been described. However, the high-frequency electrode for a medical device may be used for other high-frequency treatment tools that release high-frequency power to a biological tissue.

### EXAMPLES

Hereinafter, Examples 1 to 10 relating to the high-frequency electrode for a medical device of each of the modified examples described above will be described together with Comparative Examples 1 and 2.

Configurations of Examples 1 to 10 and Comparative Examples 1 and 2 and the results of evaluation are shown in the following Table 1.

TABLE 1

| | ELECTRODE BASE | | | | |
|---|---|---|---|---|---|
| | MATERIAL | | OXIDE | | |
| | | PARTS BY | | PARTS BY | ELECTRODE |
| | MATERIAL | MASS | MATERIAL | MASS | SHAPE |
| EXAMPLE 1 | W | 100 | ThO₂ | 2 | FLAT PLATE TYPE |
| EXAMPLE 2 | W | 100 | ThO₂ | 4 | SPATULA TYPE |
| EXAMPLE 3 | W | 100 | Y₂O₃ | 4 | ROUND ROD TYPE |
| EXAMPLE 4 | W | 100 | Y₂O₃ | 4 | ROUND ROD TYPE |
| EXAMPLE 5 | Ta | 100 | Er₂O₃ | 6 | FLAT PLATE TYPE |
| EXAMPLE 6 | Ta | 100 | Ce₂O₃ | 8 | ROUND ROD TYPE |
| EXAMPLE 7 | Nb | 100 | La₂O₃ | 10 | FLAT PLATE TYPE |
| EXAMPLE 8 | Nb | 100 | Y₂O₃ | 10 | SPATULA TYPE |
| EXAMPLE 9 | Ta | 100 | Ce₂O₃ | 8 | ROUND ROD TYPE |
| EXAMPLE 10 | Nb | 100 | TiO₂ | 10 | SPATULA TYPE |
| COMPARATIVE | W | 100 | Y₂O₃ | 4 | FLAT PLATE TYPE |
| EXAMPLE 1 | | | | | |
| COMPARATIVE | Nb | 100 | Y₂O₃ | 10 | SPATULA TYPE |
| EXAMPLE 2 | | | | | |

| | | PARTICLE | RESULTS OF EVALUATION | |
|---|---|---|---|---|
| | REPRESENTATIVE | DIAMETER | | ADHESION OF |
| | DIMENSION L3 | OF OXIDE | SURFACE | BIOLOGICAL |
| | (mm) | (μm) | ROUGHNESS | TISSUE |
| EXAMPLE 1 | 2.0 | 2-20 | A | A |
| EXAMPLE 2 | 1.0 | 2-10 | A | A |
| EXAMPLE 3 | 0.6 | 2-6 | A | A |
| EXAMPLE 4 | 0.4 | 2-4 | A | A |
| EXAMPLE 5 | 1.0 | 2-10 | A | A |
| EXAMPLE 6 | 0.4 | 2-4 | A | A |
| EXAMPLE 7 | 1.6 | 2-16 | A | A |
| EXAMPLE 8 | 1.0 | 2-10 | A | A |
| EXAMPLE 9 | 0.4 | 5-10 | B | B |
| EXAMPLE 10 | 1.0 | 2-10 | B | B |
| COMPARATIVE | 1.0 | 0.5-1.5 | C | C |
| EXAMPLE 1 | | | | |
| COMPARATIVE | 1.0 | 0.5-1.5 | C | C |
| EXAMPLE 2 | | | | |

### Example 1

The high-frequency electrode of Example 1 is an example of the high-frequency electrode 12 of the second modified example.

As shown in Table 1, in the high-frequency electrode 12 of the present example, pure metal tungsten was used as the material of the electrode base material 1A. As the material of the oxide 1B, thorium dioxide having a particle diameter of 2 nm to 20 μm (denoted as “2-20” in Table 1) was used. The oxide 1B was added in an amount of 2 parts by mass with respect to 100 parts by mass of the electrode base material 1A.

The high-frequency electrode 12 of the present example was formed by using a shaping mold for shaping a flat plate having a thickness of 2.0 mm after powdered electrode base material 1A and the oxide 1B were mixed. Powder metallurgy was used as a shaping method.

The high-frequency electrode 12 of the present example was fixed to the grip portion 2. The high-frequency electrode 12 was electrically connected to the high-frequency power supply 3. Thus, the high-frequency knife 10 of the present example was manufactured.

In the high-frequency knife 10 of the present example, the electrode shape of the effective electrode region of the high-frequency electrode 12 is a flat plate (denoted as “flat plate type” in Table 1) having a longitudinal width of 25.0 mm, a lateral width of 4.0 mm, and a thickness of 2.0 min. For this reason, the representative length L3 of the electrode shape of the high-frequency electrode 12 of the present example was 2.0 mm.

### Example 2

In the high-frequency electrode of Example 2, the particle diameter and the added amount of the oxide 1B and the electrode shape in Example 1 were changed. The electrode shape of the present example was a spatula type as shown in FIG. 5. The high-frequency electrode of the present example is an example of the high-frequency electrode 13 of the third modified example. Hereinafter, differences from Example 1 will be mainly described.

In the present example, the particle diameter of the oxide 1B was 2 μm or more and 10 μm or less. The amount of the oxide 1B added was 4 parts by mass.

The high-frequency electrode 13 of the present example was manufactured in the same manner as in Example 1 except that the shaping mold and the mixing ratio of the oxide 1B were different therefrom. The high-frequency knife 10 of the present example was manufactured using the high-frequency electrode 13 of the present example.

In the electrode shape of the effective electrode region in the high-frequency knife 10 of the present embodiment, the longitudinal width×the lateral width×the maximum thickness is 25.0 mm×2.0 mm×1.0 mm. For this reason, the representative length L3 of the electrode shape of the high-frequency electrode 13 of the present example was 1.0 mm.

### Example 3

In the high-frequency electrode of Example 3, the material, the particle diameter and the added amount of the oxide 1B and the electrode shape in Example 1 were changed. The electrode shape of the present example was a round rod type. The high-frequency electrode of the present example is an example of the high-frequency electrode 11 of the fourth modified example. Hereinafter, differences from Example 1 will be mainly described.

In the present example, yttrium oxide (Y₂O₃) having a particle diameter of 2 μm or more and 6 μm or less was used as the material of the oxide 1B. The added amount of the oxide 1B was 4 parts by mass.

The high-frequency electrode of the present example was manufactured in the same manner as in Example 1 except that the shaping mold and the material and the mixing ratio of the oxide 1B were different therefrom. The high-frequency knife 10 of the present example was manufactured using the high-frequency electrode of the present example.

In the high-frequency knife 10 of the present example, the electrode shape of the effective electrode region was set to a diameter of 0.6 mm and a length of 15.0 mm. For this reason, the representative length L3 of the electrode shape of the high-frequency electrode of the present example was 0.6 mm.

### Example 4

In the high-frequency electrode of Example 4, the diameter and the particle diameter of the oxide 1B in Example 3 were changed. The diameter of the present modified example was 0.4 mm, According to this, the particle diameter of the oxide 1B of the present modified example was set to 2 μm or more and 4 μm or less. Hereinafter, differences from Example 3 will be mainly described.

In the high-frequency knife 10 of the present example, the electrode shape of the effective electrode region was changed to a diameter of 0.4 mm and a length of 15.0 mm. For this reason, the representative length L3 of the electrode shape of the high-frequency electrode of the present example was 0.4 mm.

### Example 5

The high-frequency electrode of Example 5 is an example of the high-frequency electrode 12 of the second modified example, similarly to the high-frequency electrode of Example 1.

In the high-frequency electrode 12 of the present example, pure metal tantalum was used as the materiel of the electrode base material 14. As the material of the oxide 1B, erbium oxide (Er₂O₃) having a particle diameter of 2 μm or more and 10 μm or less was used. The oxide 1B was added in an amount of 6 parts by mass with respect to 100 parts by mass of the electrode base material 1A.

The high-frequency electrode 12 of the present example was formed by using a shaping mold for shaping a flat plate having a thickness of 1.0 mm after powdered electrode base material 1A and the oxide 1B were mixed. Powder metallurgy was used as a forming method.

The high-frequency electrode 12 of the present example was fixed to the grip portion 2. The high-frequency electrode 12 was electrically connected to the high-frequency power supply 3. Thus, the high-frequency knife 10 of the present example was manufactured.

In the high-frequency knife 10 of the present example, the electrode shape of the effective electrode region of the high-frequency electrode 12 is a flat plate having a longitudinal width of 25.0 mm, a lateral width of 3.0 mm, and a thickness of 1.0 mm. For this reason, the representative length L3 of the electrode shape of the high-frequency electrode 12 of the present example was 1.0 mm.

### Example 6

In the high-frequency electrode of Example 6, the material, the particle diameter and the added amount of the oxide 1B and the electrode shape in Example 5 were changed. The electrode shape of the present example was changed to a round rod type. The high-frequency electrode of the present example is an example of the high-frequency, electrode of the fourth modified example. Hereinafter, differences from Example 5 will be mainly described.

In the present example, cerium oxide having a particle diameter of 2 μm or more and 4 μm or less was used as the material of the oxide 1B. The added amount of the oxide 1B was 8 parts by mass.

The high-frequency electrode of the present example was manufactured in the same manner as in Example 5 except that the shaping mold, and the material and the mixing ratio of the oxide 1B were different therefrom. The high-frequency knife 10 of the present example was manufactured using the high-frequency electrode of the present example.

In the high-frequency knife 10 of the present example, the electrode shape of the effective electrode region was set to a diameter of 0.4 mm and a length of 15.0 mm. For this reason, the representative length L3 of the electrode shape of the high-frequency electrode of the present example was 0.4 mm.

### Example 7

The high-frequency electrode of Example 7 is an example of the high-frequency electrode 12 of the second modified example, similarly to the high-frequency electrode of Example 1,

In the high-frequency electrode 12 of the present example, pure metal niobium was used as the material of the electrode base material 1A. As the material of the oxide 1B, lanthanum oxide having a particle diameter of 2 μm or more and 16 μm or less was used. The oxide 1B was added in an amount of 10 parts by mass with respect to 100 parts by mass of the electrode base material 1A.

The high-frequency electrode 12 of the present example was formed by using a shaping mold for shaping a flat plate having a thickness of 1.6 mm after powdered electrode base material 1A and the oxide 1B were mixed. Powder metallurgy was used as a forming method.

The high-frequency electrode 12 of the present example was fixed to the grip portion 2. The high-frequency electrode 12 was electrically connected to the high-frequency power supply 3. Thus, the high-frequency knife 10 of the present was manufactured.

In the high-frequency knife 10 of the present example, the electrode shape of the effective electrode region of the high-frequency electrode 12 is a flat plate having a longitudinal width of 25.0 non, a lateral width of 3.0 min, and a thickness of 1.6 mm. For this reason, the representative length L3 of the electrode shape of the high-frequency electrode 12 of the present example was L6 mm.

### Example 8

In the high-frequency electrode of Example 8, the material, the particle diameter and the added amount of the oxide 1B and the electrode shape in Example 7 were changed. As the electrode shape of the high-frequency electrode of the present example, a spatula type shown in FIG. 5 was used. The high-frequency electrode of the present example is an example of the high-frequency electrode 13 of the third modified example. Hereinafter, differences from Example 7 will be mainly described.

In the present example, yttrium oxide having a particle diameter of 2 μm or more and 10 μm or less was used as the material of the oxide 1B. The oxide 1B was added in an amount of 10 parts by mass with respect to 100 parts by mass of the electrode base material 1A.

The high-frequency electrode 13 of the present example was manufactured in the same manner as in Example 7 except that the shaping mold, and the material and mixing ratio of the oxide 1B were different therefrom. The high-frequency knife 10 of the present example was manufactured using the high-frequency electrode 13 of the present example.

In the electrode shape of the effective electrode region in the high-frequency knife 10 of the present example, the longitudinal width×the lateral width×the maximum thickness is 25.0 mm×2.0 mm×1.0 mm. For this reason, the representative length L3 of the electrode shape of the high-frequency electrode 13 of the present example was 1.0 mm.

### Example 9

In the high-frequency electrode of Example 9, the material, the particle diameter and the added amount of the electrode base material 1A and the electrode shape in Example 5 were changed. The high-frequency electrode of the present example is an example of the high-frequency electrode of the fourth modified example. Hereinafter, differences from Example 5 will be mainly described.

In the present example, cerium oxide having a particle diameter of 5 μm or more and 10 μm or less was used as the material of the oxide 1B. The added amount of the oxide 1B was 8 parts by mass.

The high-frequency electrode of the present example was manufactured in the same manner as in Example 5 except that the shaping mold, and the material and the mixing ratio of the oxide 1B were different therefrom. The high-frequency knife 10 of the present example was manufactured using the high-frequency electrode of the present example.

In the high-frequency knife 10 of the present example, the electrode shape of the effective electrode region was set to a diameter of 0.4 mm and a length of 15.0 mm. For this reason, the representative length L3 of the electrode shape of the high-frequency electrode of the present example was 0.4 mm.

### Example 10

In the high-frequency electrode of Example 10, the material of the oxide 1B in Example 8 was changed. As the electrode shape of the high-frequency electrode of this example, a spatula type shown in FIG. 5 was used. The high-frequency electrode of the present example is an example of the high-frequency electrode 13 of the third modified example. Hereinafter, differences from Example 8 will be mainly described.

In the present example, titanium oxide having a particle diameter of 2 μm or more and 10 μm or less was used as the material of the oxide 1B. The oxide 1B was added in an amount of 10 parts by mass with respect to 100 parts by mass of the electrode base material 1A.

The high-frequency electrode 13 of the present example was manufactured in the same manner as in Example 8 except that the material of the oxide 1B was different therefrom. The high-frequency knife 10 of the present example was manufactured using the high-frequency electrode 13 of the present example.

In the electrode shape of the effective electrode region in the high-frequency knife 10 of the present example, the longitudinal width×the lateral width×the maximum thickness is 25.0 mm×2.0 mm×1.0 mm. For this reason, the representative length L3 of the electrode shape of the high-frequency electrode 13 of the present example was 1.0 mm.

### Comparative Example 1

In the high-frequency electrode of Comparative Example 1, the material and the mixing ratio of the oxide 1B in Example 1 were changed, and the representative length L3 was also changed. Hereinafter, differences from Example 1 will be mainly described.

As the oxide 1B of the present comparative example, yttrium oxide having a particle diameter of 0.5 μm or more and 1.5 μm or less was used. The oxide 1B was added in an amount of 4 parts by mass with respect to 100 parts by mass of the electrode base material 1A.

In the electrode shape of the effective electrode region in the high-frequency knife of the present comparative example, the longitudinal width×the lateral width×the maximum thickness was 25.0 mm×3.0 mm×1.0 mm. For this reason, the representative length L3 of the electrode shape of the high-frequency electrode of the present comparative example was 1.0 mm.

### Comparative Example 2

In the high-frequency electrode of Comparative Example 2, the particle diameter of the oxide 1B in Example 8 was changed to 0.5 μm or more and 1.5 μm or less.

### [Method of Evaluation]

In order to evaluate the high-frequency electrodes of the respective examples and the comparative examples, treatment operations using the high-frequency knife including the high-frequency electrodes was repeated. A pig stomach was used as a treatment subject. One treatment operation included an incision operation and a hemostatic operation. In the incision operation, the subject was incised 70 mm in length. However, the hemostatic operation did not actually perform hemostasis of the treatment subject, but the high frequency used for hemostasis was applied by pressing the high-frequency electrode on the treatment subject for a time period necessary for hemostasis. These treatment operations were repeated 100 times for each high-frequency electrode (repeated treatment tests).

After the repeated treatment tests, “Surface roughness” and “Adhesion of biological tissue” were evaluated for each high-frequency electrode.

The “Surface roughness” was evaluated based on measured values of the maximum height Ry (JIS B 0601-1994) of the electrode surface using a laser microscope. When the maximum height Ry was less than 5 μm, it was evaluated as “very good” (denoted by “A” in Table 1), 5 μm or more and less than 10 μm was evaluated “good” (denoted by “B” in Table 1), and 10 μm or ore was evaluated as “no good” (denoted by “C” in Table 1).”

The “Adhesion of biological tissue” was evaluated based on measured values of an adhesion area, on which the biological tissue adhered on the electrode surface of the effective electrode region. An optical microscope was used as an evaluation device. When the adhesion area of the biological tissue was less than 5% of a surface area of the electrode surface of the effective electrode region, it was evaluated as “very good” (denoted by “A” in Table 1), 5% or more and less than 10% was evaluated “good” (denoted by “B” in Table 1), and 10% or more was evaluated as “no good” (denoted by “C” in Table 1).

### [Results of Evaluation]

As shown in Table 1, in Examples 1 to 8, the results of the evaluations of “Surface roughness” and “Adhesion of biological tissue” were all “very good.” Also, in Examples 9 to 10, the results of the evaluations of “Surface roughness” and “Adhesion of biological tissue” were all “good.”

In contrast to this, in Comparative Examples 1 and 2, the results of the evaluations of “Surface roughness” and “Adhesion of biological tissue” were all “no good.” In each of Comparative Examples 1 and 2, since the particle diameter of the oxide was less than 2 μm, the cooling effect obtained by the oxide was considered too small.

While the preferred embodiment, the modifications, and the examples of the invention have been described and illustrated above, it should be understood that these are exemplary of the invention and are not to be considered as limiting. Additions, omissions, substitutions, and other modifications can be made without departing from the scope of the present invention. Accordingly, the invention is not to be considered as being limited by the foregoing description, and is only limited by the scope of the appended claims,

What is claimed is:

1. A high-frequency electrode for a medical device, the high-frequency electrode comprising:
an electrode base material made of a metal or an alloy, the metal or the alloy having a melting point of 2000° C. or higher; and
an oxide added to the electrode base material, the oxide having a particle diameter of 2 μm or more.

2. The high-frequency electrode for a medical device according to claim 1,
wherein a particle diameter of the oxide is equal to or less than 1/100 of a representative length of an electrode shape by an effective electrode region a narrow direction thereof.

3. The high-frequency electrode for a medical device according to claim 1,
wherein the electrode base material contains one or more metallic elements selected from a group consisting of tungsten (W), niobium (Nb), and tantalum (Ta).

4. The high-frequency electrode for a medical device according to claim 1,
wherein a standard energy of formation in the standard state (298.15 K and 105 Pa) of the oxide is equal to or less than −240 kcal/mol.

5. A medical device comprising the high-frequency electrode for a medical device according to claim 1.
